(19) United States
(12) Patent Application Publication
(10) Pub. No.: US 2025/0312186 A1
(43) Pub. Date: Oct. 9, 2025

(54) NON-HORMONAL CONTRACEPTIVE AND METHOD OF APPLICATION

(71) Applicant: Elizabeth Irle, Portland, OR (US)

(72) Inventor: Elizabeth Irle, Portland, OR (US)

(21) Appl. No.: 18/955,611

(22) Filed: Nov. 21, 2024

Related U.S. Application Data
(60) Provisional application No. 63/603,256, filed on Nov. 28, 2023.

(51) Int. Cl. A61F 6/14

(52) U.S. Cl. CPC A61F 6/146

(57) ABSTRACT

Implementations of the present disclosure may include a non-hormonal contraceptive, including a body formed of one or more non-porous, biocompatible materials. In some implementations, the body has a length in a range from 1 centimeter to 31 centimeters. In some implementations, the body has a width in a range from 0.5 centimeters to 10 centimeters. Various implementations may also include a copper inlay on the body, the copper inlay having an exposed surface area in a range from 200 square millimeters to 1000 square millimeters.

### CROSS-REFERENCES

This application claims priority to U.S. Provisional Patent Application No. 63/603,256 entitled “NON-HORMONAL CONTRACEPTIVE AND METHOD OF APPLICATION” of Elizabeth Marie Irle, filed Nov. 28, 2023, the entirety of which is incorporated herein by reference.

### TECHNICAL FIELD

This disclosure relates to reproductive health and more specifically non-hormonal contraceptives.

### BACKGROUND

Reproductive health is a key aspect of public health, and include the comprehensive state of physical, mental, and social well-being in aspects related to the reproductive system and its functions. It encompasses a spectrum of services, interventions, and information that contribute to the optimal functioning of the reproductive system. This includes access to contraception, family planning services, prenatal care, safe and legal abortion services, as well as prevention and management of sexually transmitted infections. Furthermore, reproductive health considerations extend beyond the clinical realm and encompass sociocultural factors and the promotion of sexual and reproductive rights. A robust framework for reproductive health is essential to ensure individuals and communities have the necessary resources and support to make informed decisions about their reproductive lives, ultimately contributing to improved overall public health outcomes. Contraception is a critical aspect of reproductive health, particularly for women. However, current forms of contraception may be difficult to implement effectively or have serious side effects, particularly for women. Thus, improvements are needed.

### SUMMARY

Implementations of the present disclosure may include a non-hormonal contraceptive, including a body formed of one or more non-porous, biocompatible materials. The body may have a length in a range from 1 centimeter to 31 centimeters, and/or a width in a range from 0.5 centimeters to 10 centimeters. Various implementations may include a copper inlay on the body. The copper inlay may have an exposed surface area in a range from 200 square millimeters to 700 square millimeters. At least a portion of the body may be spherical or spherocylindrical. In some implementations, the body may be egg-shaped.

In some implementations, the body may include an applicator end and a manipulator end opposite the applicator end. The copper inlay may be on the applicator end of the body. The manipulator end of the body may include one or more of a string, a cord, or a handle. In some implementations, the manipulator end and the applicator end may be comprised of a same material. Or, the manipulator end and the applicator end may be comprised of different materials. In various implementations, the applicator end may be spherical or spherocylindrical. The manipulator end may be cylindrical.

In some implementations, the body may include one or more of a synthetic material and a natural material. For example, the body may be comprised of a synthetic material such as silicone, glass, and/or stainless steel. As another example, the body may be comprised in part or in whole of a natural material such as jade, quartz, a feldspar such as labradorite, and/or a beryl such as heliodor. In general, the natural material may include a stone and/or a crystal, of which the foregoing are examples.

The copper inlay may have a width in a range from 0.07 millimeters to 23 millimeters. The copper inlay may have a uniform width or a non-uniform width. In some implementations, the copper inlay may include two or more sections not connected to each other. At least a portion of an outer surface of the copper inlay may be flush with an outer surface of the body. The outer surface of the copper inlay may be raised above the outer surface of the body. The outer surface of the copper inlay may be sunk below the outer surface of the body. In some implementations, the copper inlay may be attached to the body by a biocompatible adhesive.

Implementations of the present disclosure may include a non-hormonal contraception method. The method may include providing a non-hormonal contraceptive, such as the various examples described above, and inserting the apparatus into a vaginal canal of a person. The apparatus may be inserted such that at least a portion of the copper inlay may be within the vaginal canal. The method may include maintaining the non-hormonal contraceptive in the vaginal canal for a period in a range from 1 minute to 30 minutes. The method may include withdrawing the apparatus from the vaginal canal. Various implementations may include inserting the apparatus into the vaginal canal within 72 hours before or after intercourse, within 48 hours before or after intercourse, within 36 hours before or after intercourse, within 24 hours before or after intercourse, within 12 hours before or after intercourse, or during intercourse.

The non-hormonal contraceptive used in the method may include a body formed of one or more non-porous, biocompatible materials. The body may have a length in a range from 1 centimeter to 31 centimeters and/or a width in a range from 0.5 centimeters to 10 centimeters. The non-hormonal contraception may also include a copper inlay on the body, the copper inlay having an exposed surface area in a range from 200 square millimeters to 1000 square millimeters.

### BRIEF DESCRIPTION OF THE DRAWINGS

The present description will be understood more fully when viewed in conjunction with the accompanying drawings of various examples of a non-hormonal contraceptive and method of application. The description is not meant to limit the non-hormonal contraceptive or method of application to the specific examples. Rather, the specific examples depicted and described are provided for explanation and understanding of the non-hormonal contraceptive and method of application. Throughout the description the drawings may be referred to as drawings, figures, and/or FIGS.

FIG. 1 depicts a non-hormonal contraceptive, according to some implementations of the present disclosure.

FIG. 2 illustrates an egg-shaped non-hormonal contraceptive, according to some implementations of the present disclosure.

FIG. 3 illustrates a wand-shaped non-hormonal contraceptive, according to some implementations of the present disclosure.

FIG. 4 illustrates a bullet-shaped non-hormonal contraceptive, according to some implementations of the present disclosure.

FIGS. 5A-B illustrate a copper-plated non-hormonal contraceptive 500, according to some implementations of the present disclosure.

FIG. 6 illustrates a bullet-shaped and copper-plated non-hormonal contraceptive 600, according to some implementations.

FIGS. 7A-C depict cross-sections of various implementations of a non-hormonal contraceptive.

FIG. 8 is a flowchart that describes a non-hormonal contraception method, according to some implementations of the present disclosure.

### DETAILED DESCRIPTION

A non-hormonal contraceptive and method of application as disclosed herein will become better understood through a review of the following detailed description in conjunction with the figures. The detailed description and figures provide merely examples of the various implementations of the non-hormonal contraceptive and method of application. Many variations are contemplated for different applications and design considerations; however, for the sake of brevity and clarity, all the contemplated variations may not be individually described in the following detailed description. Those skilled in the art will understand how the disclosed examples may be varied, modified, and altered and not depart in substance from the scope of the examples described herein.

Conventional forms of contraception may include abstinence, barriers such as condoms and diaphragms, hormonal contraceptives such as birth control pills and hormonal intrauterine devices (IUDs), and non-hormonal contraceptives such as copper IUDs, spermicides, and tubal ligation. These conventional contraceptives, while abundant, create a host of problems in women's and reproductive health. For example, abstinence, while 100% effective when employed, may deprive a person of certain aspects of intimacy that are critical to physical and mental wellbeing. Barriers are highly susceptible to improper use and/or physical failure. Hormonal contraceptives are shown to have side effects such as headaches, nausea, sore breasts, thrush, mood swings, reduced sexual desire, and/or thrombosis. Other side effects may include structural changes to the brain regions responsible for memory, emotion, attraction, and mental health. Hormonal birth control has also been found to increase the levels of cortisol in women using it. This may alter the brain, the bloodstream, and immune system and depresses the digestive and reproductive systems. IUDs can lead to increased period bleeding, irregular periods, amenorrhea, worsened menstrual cramps, ovarian cysts, or, in severe cases, perforation of the uterine wall, temporary infertility, and/or permanent infertility.

Conventional approaches to contraception, therefore, tend to be associated with some burden, typically born by women, that may decrease the overall positive health impacts of intimacy and intercourse. A non-hormonal contraceptive and method of application, as disclosed herein, however, may eliminate many, if not all of the negative impacts contraceptives have on reproductive and women's health. Notably, the non-hormonal contraceptive and method of application disclosed herein may actually increase and/or improve pleasure associated with intimacy and intercourse.

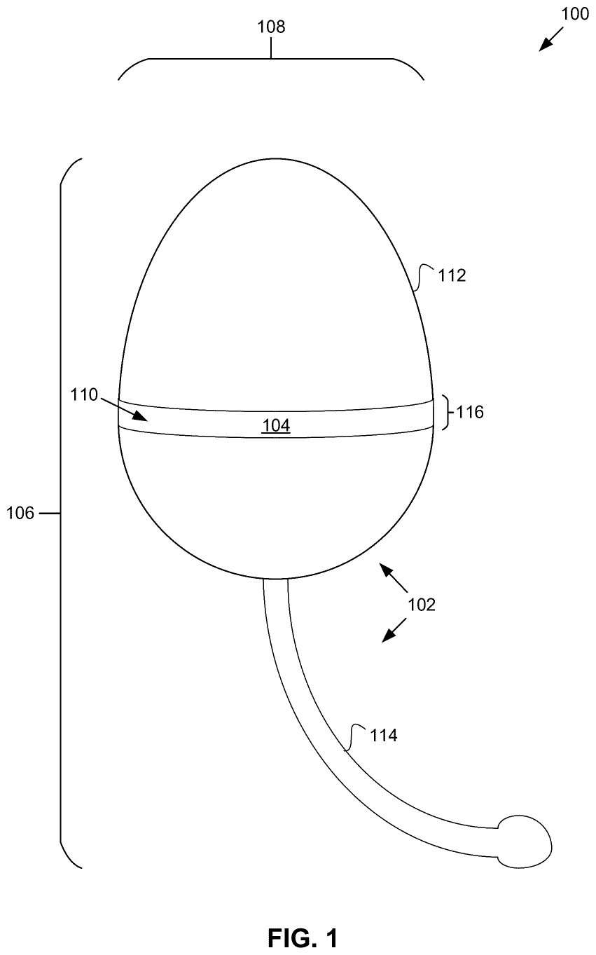

FIG. 1 depicts a non-hormonal contraceptive 100, according to some implementations of the present disclosure. The non-hormonal contraceptive 100 may include a body 102 with a copper inlay 104. The body 102 may have a length 106 in a range from 1 centimeter to 31 centimeters. The body 102 may have a width 108 in a range from 0.5 centimeters to 10 centimeters. More generally, the body 102 may have dimensions and/or volume such that the non-hormonal contraceptive 100 or a portion of the non-hormonal contraceptive 100 may be inserted into a vaginal canal of a person. The copper inlay 104 may have an exposed surface area 110 in a range from 200 square millimeters to 1000 square millimeters. A width 116 of the copper inlay 104 may be in a range from 0.07 millimeters to 80 millimeters.

In some implementations, the body 102 may include an applicator end or portion 112 and a manipulator end or portion 114. The manipulator end 114 may be opposite the applicator end 112. The copper inlay 104 may be on the applicator end 112 of the body 102. The applicator end 112 may be used to expose the copper inlay 104 to a vaginal canal of a person. The manipulator end 114 may be used to insert the applicator end 112 into the vaginal canal and/or to withdraw the applicator end 112 from the vaginal canal. In various implementations, the manipulator end 114 may include one or more of a string, a cord, or a handle.

At least a portion of the body 102 may be spherical or spherocylindrical. In some implementations, the body 102 may be egg-shaped. The body 102 may be formed of one or more non-porous, solid materials. The body 102 may be formed of one or more biocompatible materials. In some implementations, the body 102 may be comprised of a synthetic material, a natural material, or a synthetic material and a natural material. The body 102 may be comprised of two or more synthetic materials, two or more natural materials, or a combination of synthetic and natural materials. The synthetic material may, for example, include silicone, glass, and/or stainless steel. The natural material may, for example, include jade, quartz, or another naturally-occurring, non-porous stone.

As a specific example, the non-hormonal contraceptive 100 may include a body 102 with the applicator end 112 and the manipulator end 114. The applicator end 112 may have a length of approximately 5 centimeters, and the manipulator end 114 may have a length of approximately 8 centimeters. The length 106 of the body 102 may be approximately 13 cm. The applicator end 112 may have a width of approximately 4 centimeters, such as at its widest width. The applicator end 112 may be formed of quartz and may have a smooth, curved surface. The applicator end 112 may be polished and/or may include a sealant or other veneer. The applicator end 112 may be egg-shaped. The copper inlay 104 may be inlaid into the surface of the applicator end 112 and may form a band around the applicator end 112 with the exposed surface area 110 of the copper inlay 104 being approximately 350 square millimeters. The width 116 of the copper inlay 104 may be approximately 2.8 millimeters. The manipulator end 114 may be a cord formed of silicone and attached to the applicator end 112. For example, a portion of the manipulator end 114 may be inserted into a hole in the applicator end 112 and secured to the applicator end 112 by an adhesive, tension fitting, machine pressing, solder, heat bonding, and so forth.

As another specific example, the applicator end 112 may be formed of jade and may be spherical. A diameter of the applicator end 112 may be approximately 3 centimeters. The copper inlay 104 may be inlaid into the surface of the applicator end 112 and may form a band around the applicator end 112 with the exposed surface area 110 of the copper inlay 104 being approximately 450 square millimeters. The width 116 of the copper inlay 104 may be approximately 3.6 millimeters. The manipulator end 114 may be a cord formed of silicone and attached to the applicator end 112. The manipulator end 114 may have a length of approximately 10 centimeters, making the length 106 of the body approximately 14 centimeters.

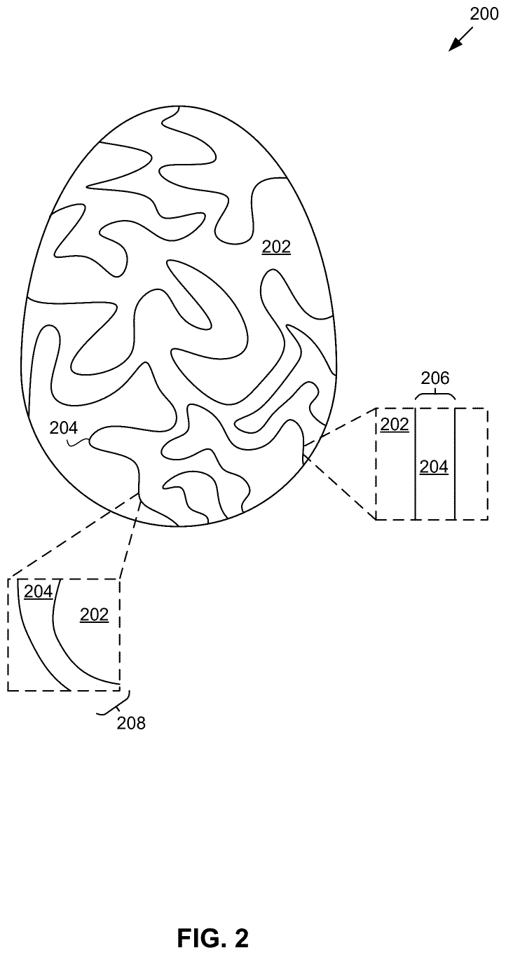

FIG. 2 illustrates an egg-shaped non-hormonal contraceptive 200, according to some implementations of the present disclosure. The egg-shaped non-hormonal contraceptive 200 may include a body 202 and a copper inlay 204. The copper inlay 204 may be a copper wire having a narrow diameter, such as in a range from 0.07 millimeters to 1 millimeter. The copper inlay 204 may be a copper sheet or cutout. The copper inlay 204 may be set into the surface of the body 202 and form a pattern or design on the body 202. The copper inlay 204 may have a uniform width, e.g., as shown at 206. The copper inlay 204 may have a non-uniform width, e.g., as shown at 208.

In various implementations, such as the example depicted in FIG. 2, the non-hormonal contraceptive may not include separate applicator and manipulator ends. The body 202 may be formed of a single material or a combination of materials. In such implementations, application of the non-hormonal contraceptive may include insertion of the entire body into the vaginal canal. The person may retain the non-hormonal contraceptive in the vaginal canal by contracting their pelvic floor muscles. The person may retain the non-hormonal contraceptive in the vaginal canal by laying down. The non-hormonal contraceptive may be withdrawn from the vaginal canal by relaxing the pelvic floor muscles and/or using gravity. The non-hormonal contraceptive may be withdrawn from the vaginal canal by the user pushing it out with the user's pelvic floor muscles.

As a specific example, the body 202 may be formed of a non-porous material such as stainless steel. The outer surface of the body 202 may be etched and the copper inlay 204 may be secured in the etching. The body 202 may have a length of approximately 7 centimeters. A width of the body 202 may be approximately 5 centimeters, e.g., at a widest point of the body. The copper inlay 204 may have a uniform width of approximately 5 millimeters and a length of approximately 60 millimeters. An exposed surface area of the copper inlay 204 may be approximately 300 square millimeters.

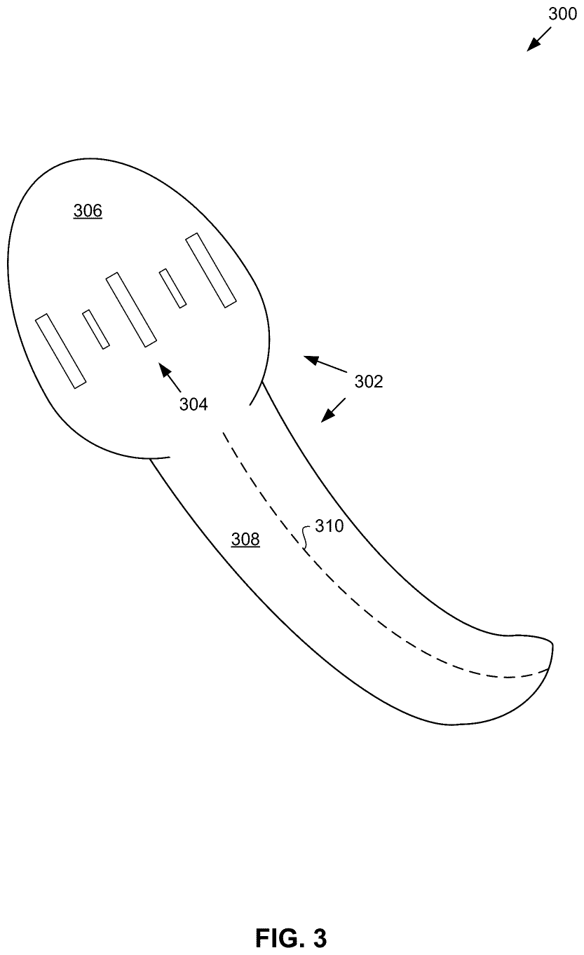

FIG. 3 illustrates a wand-shaped non-hormonal contraceptive 300, according to some implementations of the present disclosure. The wand-shaped non-hormonal contraceptive 300 may include a body 302 and a copper inlay 304 set into an outer surface of the body 302. The copper inlay 304 may include two or more sections that are not connected to each other. The body 302 may include an applicator end 306 and a manipulator end 308. The applicator end 306 and the manipulator end 308 may be formed of the same material. For example, the body 302 may be a continuous unit from the applicator end 306 to the manipulator end 308.

As a specific example, the body 302 may be chiseled from a single piece of non-porous stone. The applicator end 306 may be formed into an egg-shape or a spherical shape. The manipulator end 308 may be formed into a cylindrical shape or a semi-cylindrical shape. The manipulator end 308 may be curved. In some implementations, the manipulator end 308 may be u-shaped. The applicator end 306 may have a length of approximately 4 centimeters. The manipulator end 308 may have a curved length 310 of approximately 12 centimeters. The overall length of the of the body 302 may be approximately 15 centimeters. The copper inlay 304 may include multiple sections of varying length and/or width set into an outer surface of the applicator end 306.

As another example, the manipulator end 308 may be j-shaped. The applicator end 306 may be attached to a short side of the manipulator end 308. The curved length 310 of the manipulator end 308 may be approximately 27 centimeters, and the overall length of the body 302 may be approximately 19 centimeters. A curved portion of the manipulator end 308 may have a radius of curvature of approximately 4 centimeters.

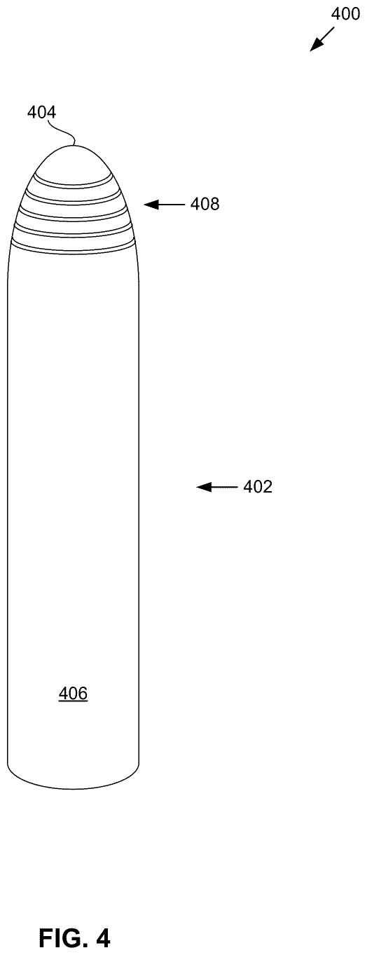

FIG. 4 illustrates a bullet-shaped non-hormonal contraceptive 400, according to some implementations of the present disclosure. The bullet-shaped non-hormonal contraceptive 400 may include a body 402 with an applicator end 404 and a manipulator end 406. The bullet-shaped non-hormonal contraceptive 400 may also include a copper inlay 408 set into an outer surface of the body 402 at the applicator end 404. The body 402 may have a length of approximately 6 centimeters and a width (e.g., a widest width) of approximately 1 centimeter. An exposed surface area of the copper inlay 408 may be approximately 250 square centimeters. The copper inlay 408 may include multiple rings around the body 402 at the applicator end 404.

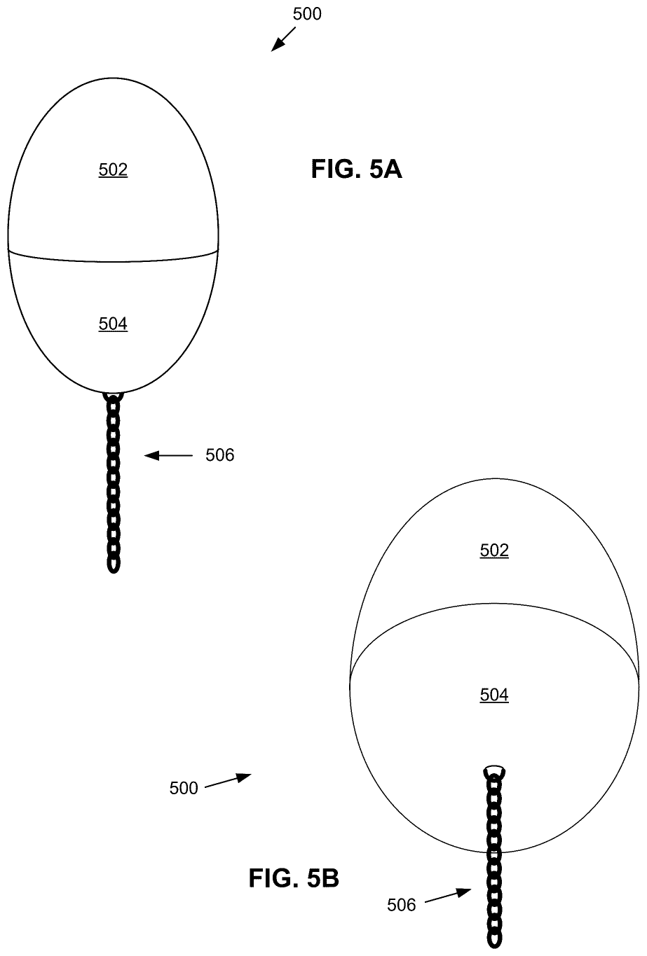

FIGS. 5A-B illustrate a copper-plated non-hormonal contraceptive 500, according to some implementations of the present disclosure. The copper-plated non-hormonal contraceptive 500 may include a body 502 and copper plating 504. The copper plating 504 may be formed and/or molded on an outer surface of the body 502. The copper plating 504 may be attached to the outer surface of the body 502. A chain 506 may be coupled to the body 502 and/or the copper plating 504. For example, the chain 506 may be soldered to the copper plating 504. The copper plating 504 may include a molded loop to which the chain 506 may be attached. In various implementations, the chain 506 may be made of stainless steel.

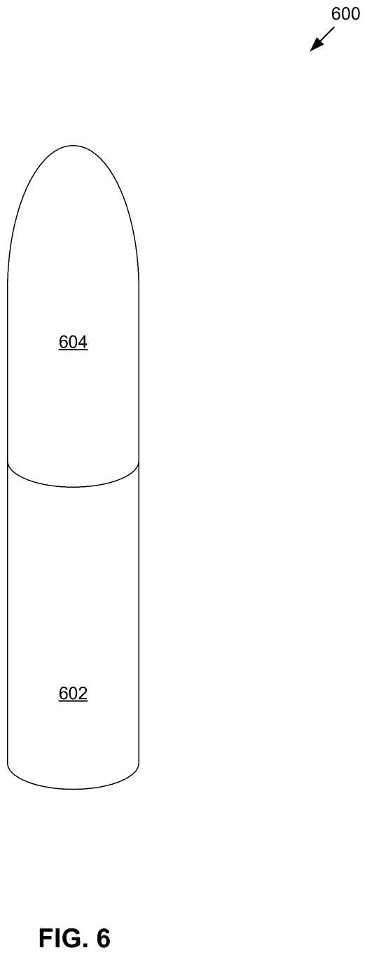

FIG. 6 illustrates a bullet-shaped and copper-plated non-hormonal contraceptive 600, according to some implementations. The bullet-shaped and copper-plated non-hormonal contraceptive 600 may include a body 602 and copper plating 604. The copper plating 604 may be formed and/or molded on an outer surface of the body 602.

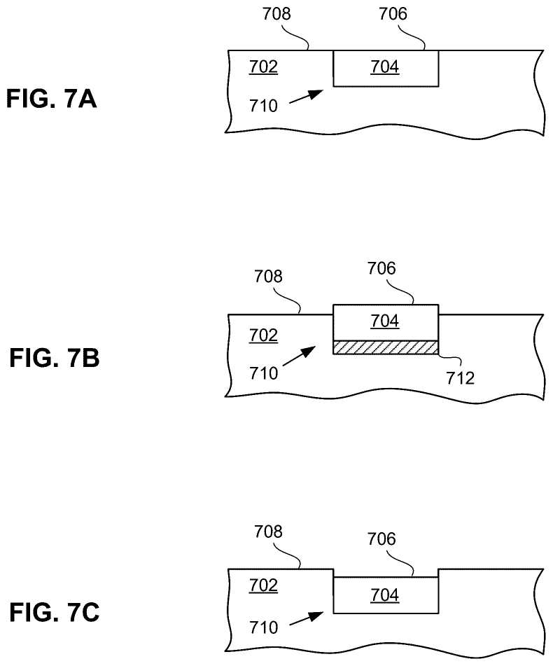

FIGS. 7A-C depict cross-sections of various implementations of a non-hormonal contraceptive. In general, the non-hormonal contraceptive may include a body 702 and a copper inlay 704. In some implementations, at least a portion of an outer surface 706 of the copper inlay 704 may be flush with an outer surface 708 of the body 702. In some implementations, at least a portion of the outer surface 706 of the copper inlay 704 may be raised above the outer surface 708 of the body 702. In various implementations, at least a portion of the outer surface 706 may be sunk below the outer surface 708 of the body 702.

In some implementations, the copper inlay 704 may be attached to the body by friction. For example, the copper inlay 704 may be hammered into a recess 710 in the body 702. This may increase the friction between the copper inlay 704 and the body 702. In some implementations, the copper inlay 704 may be attached to the body 702 by a biocompatible adhesive 712. The biocompatible adhesive 712 may be non-toxic to humans and/or suitable for use in the presence of bodily fluids. The biocompatible adhesive 712 may include one or more of an epoxy, a silicone adhesive, a cyanoacrylate adhesive, and/or an epoxy-polyurethane blend.

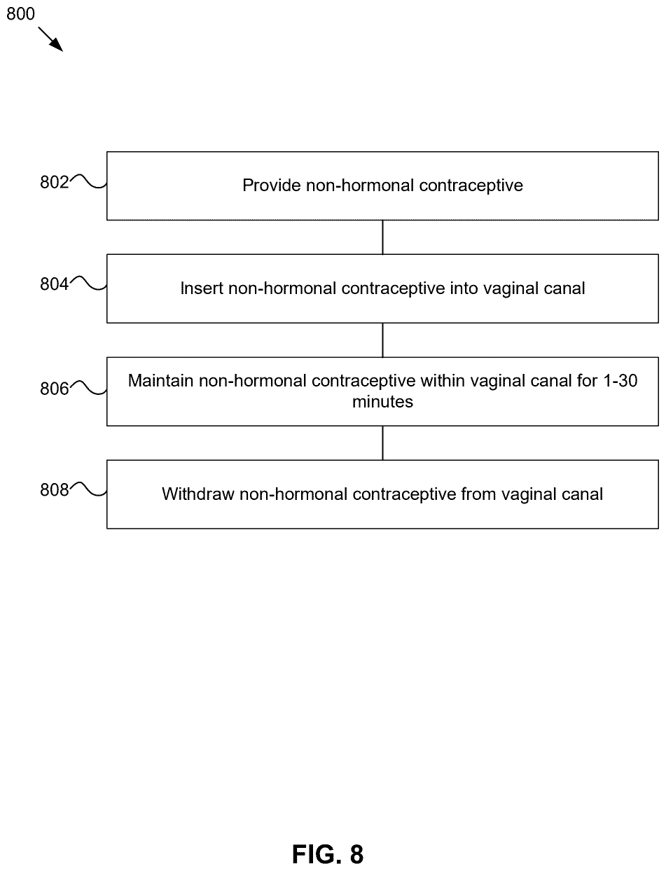

FIG. 8 is a flowchart that describes a non-hormonal contraception method 800, according to some implementations of the present disclosure. In some implementations, at 802, the method may include providing a non-hormonal contraceptive such as an implementation of the non-hormonal contraceptive described above. The non-hormonal contraceptive may include a body with a copper inlay. The body may be formed of one or more non-porous, biocompatible materials. The body may have a length in a range from 1 centimeter to 31 centimeters. The body may have a width in a range from 0.5 centimeters to 10 centimeters. The copper inlay may have an exposed surface area in a range from 200 square millimeters to 700 square millimeters.

At 804, the method may include inserting the non-hormonal contraceptive into a vaginal canal of a person. The non-hormonal contraceptive may be inserted into the vaginal canal such that at least a portion of the copper inlay may be within the vaginal canal. In some implementations, the method may include inserting the non-hormonal contraceptive into the vaginal canal such that at least 200 square millimeters of surface area of the copper inlay is within the vaginal canal. In some implementations, the method may include inserting the non-hormonal contraceptive into the vaginal canal such that at least 250 square millimeters of surface area of the copper inlay is within the vaginal canal. In some implementations, the method may include inserting the non-hormonal contraceptive into the vaginal canal such that at least 300 square millimeters, at least 350 square millimeters, at least 400 square millimeters, at least 450 square millimeters, at least 500 square millimeters, at least 600 square millimeters, at least 800 square millimeters, or at least 1000 square millimeters of surface area of the copper inlay is within the vaginal canal.

At 806, the method may include maintaining the non-hormonal contraceptive in the vaginal canal for a period in a range from 1 minute to 30 minutes. For implementations of the non-hormonal contraceptive with a larger surface area of exposed copper, the device may be maintained in the vaginal canal for a shorter period for implementations with larger exposed surface areas of copper. For example, an implementation with 750 square millimeters of exposed copper, with all or a substantial portion of the exposed copper within the vaginal canal, may be similarly effective at contraception when maintained in the vaginal canal for 1 to 5 minutes as implementations with 350 square millimeters of exposed copper when maintained in the vaginal canal for 7 to 15 minutes.

At 808, the non-hormonal contraception method may include withdrawing the non-hormonal contraceptive from the vaginal canal. In various implementations, the non-hormonal contraceptive may be inserted into the vaginal canal within 72 hours before or after intercourse, within 48 hours before or after intercourse, within 36 hours before or after intercourse, within 24 hours before or after intercourse, within 12 hours before or after intercourse, and/or during intercourse. In some implementations, the method may be effective at preventing pregnancy if performed within 5 days after intercourse.

A feature illustrated in one of the figures may be the same as or similar to a feature illustrated in another of the figures. Similarly, a feature described in connection with one of the figures may be the same as or similar to a feature described in connection with another of the figures. The same or similar features may be noted by the same or similar reference characters unless expressly described otherwise. Additionally, the description of a particular figure may refer to a feature not shown in the particular figure. The feature may be illustrated in and/or further described in connection with another figure.

Elements of processes (i.e., methods) described herein may be executed in one or more ways such as by a human, by a processing device, by mechanisms operating automatically or under human control, and so forth. Additionally, although various elements of a process may be depicted in the figures in a particular order, the elements of the process may be performed in one or more different orders without departing from the substance and spirit of the disclosure herein.

The foregoing description sets forth numerous specific details such as examples of specific components, methods, and so forth, in order to provide a good understanding of several implementations. It will be apparent to one skilled in the art, however, that at least some implementations may be practiced without these specific details. In other instances, well-known components or methods are not described in detail or are presented in simple block diagram format in order to avoid unnecessarily obscuring the present implementations. Thus, the specific details set forth above are merely examples. Particular implementations may vary from these details and still be contemplated to be within the scope of this disclosure.

Related elements in the examples and/or implementations described herein may be identical, similar, or dissimilar in different examples. For the sake of brevity and clarity, related elements may not be redundantly explained. Instead, the use of a same, similar, and/or related element names and/or reference characters may cue the reader that an element with a given name and/or associated reference character may be similar to another related element with the same, similar, and/or related element name and/or reference character in an example explained elsewhere herein. Elements specific to a given example may be described regarding that particular example. A person having ordinary skill in the art will understand that a given element need not be the same and/or similar to the specific portrayal of a related element in any given figure or example in order to share features of the related element.

It is to be understood that the foregoing disclosure is intended to be illustrative and not restrictive. Many other implementations will be apparent to those of skill in the art upon reading and understanding the above disclosure. The scope of the present implementations should, therefore, be determined with reference to the appended claims, along with the full scope of equivalents to which such claims are entitled.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements.

As used herein “same” means sharing all features and “similar” means sharing a substantial number of features or sharing materially important features even if a substantial number of features are not shared. As used herein “may” should be interpreted in a permissive sense and should not be interpreted in an indefinite sense. Additionally, use of “is” regarding examples, elements, and/or features should be interpreted to be definite only regarding a specific example and should not be interpreted as definite regarding every other example. Furthermore, references to “the disclosure” and/or “this disclosure” refer to the entirety of the writings of this document and the entirety of the accompanying illustrations, which extends to all the writings of each subsection of this document, including the Title, Technical Field, Background, Brief description of the Drawings, Detailed Description, Claims, Abstract, and any other document and/or resource incorporated herein by reference.

As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. An example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not have been redundantly labeled. A person having ordinary skill in the art will recognize based on the disclosure herein redundant and/or duplicated elements of the same FIG. Despite this, redundant labeling may be included where helpful in clarifying the structure of the depicted examples.

The Applicant(s) reserves the right to submit claims directed to combinations and sub-combinations of the disclosed examples that are believed to be novel and non-obvious. Examples embodied in other combinations and sub-combinations of features, functions, elements and/or properties may be claimed through amendment of those claims or presentation of new claims in the present application or in a related application. Such amended or new claims, whether they are directed to the same example or a different example and whether they are different, broader, narrower, or equal in scope to the original claims, are to be considered within the subject matter of the examples described herein.

We claim:

1. A non-hormonal contraceptive, comprising:
a body formed of one or more non-porous, biocompatible materials, wherein the body has a length in a range from 1 centimeter to 31 centimeters, and wherein the body has a width in a range from 0.5 centimeters to 10 centimeters; and
a copper inlay on the body, the copper inlay having an exposed surface area in a range from 200 square millimeters to 1000 square millimeters.

2. The non-hormonal contraceptive of claim 1, wherein at least a portion of the body is spherical or spherocylindrical.

3. The non-hormonal contraceptive of claim 2, wherein the body is egg-shaped.

4. The non-hormonal contraceptive of claim 1, wherein the body comprises an applicator end and a manipulator end opposite the applicator end.

5. The non-hormonal contraceptive of claim 4, wherein the copper inlay is on the applicator end of the body.

6. The non-hormonal contraceptive of claim 4, wherein the manipulator end of the body comprises one or more of a string, a cord, or a handle.

7. The non-hormonal contraceptive of claim 4, wherein the manipulator end and the applicator end are comprised of a same material.

8. The non-hormonal contraceptive of claim 4, wherein the manipulator end and the applicator end are comprised of different materials.

9. The non-hormonal contraceptive of claim 4, wherein the manipulator end is spherical or spherocylindrical.

10. The non-hormonal contraceptive of claim 4, wherein the applicator end is cylindrical.

11. The non-hormonal contraceptive of claim 1, wherein the body is comprised of one or more of a synthetic material and a natural material.

12. The non-hormonal contraceptive of claim 11, wherein the synthetic material comprises one or more of silicone, glass, and stainless steel.

13. The non-hormonal contraceptive of claim 11, wherein the natural material comprises one or more of stone or crystal.

14. The non-hormonal contraceptive of claim 1, wherein the copper inlay has a width in a range from 0.07 millimeters to 23 millimeters.

15. The non-hormonal contraceptive of claim 1, wherein the copper inlay has a uniform width or a non-uniform width.

16. The non-hormonal contraceptive of claim 1, wherein the copper inlay comprises two or more sections not connected to each other.

17. The non-hormonal contraceptive of claim 1, wherein at least a portion of an outer surface of the copper inlay is flush with an outer surface of the body, raised above the outer surface of the body, or sunk below the outer surface of the body.

18. The non-hormonal contraceptive of claim 1, wherein the copper inlay is attached to the body by one or more of a biocompatible adhesive, tension fitting, machine pressing, solder, and heat bonding.

19. A non-hormonal contraception method, comprising:
providing a non-hormonal contraceptive that comprises:
a body formed of one or more non-porous, biocompatible materials, wherein the body has a length in a range from 1 centimeter to 31 centimeters, and wherein the body has a width in a range from 0.5 centimeters to 10 centimeters; and
a copper inlay on the body, the copper inlay having an exposed surface area in a range from 200 square millimeters to 700 square millimeters;
inserting the non-hormonal contraceptive into a vaginal canal of a person such that at least a portion of the copper inlay is within the vaginal canal;
maintaining the non-hormonal contraceptive in the vaginal canal for a period in a range from 1 minute to 30 minutes; and
withdrawing the non-hormonal contraceptive from the vaginal canal.

20. The non-hormonal contraception method of claim 19, wherein the non-hormonal contraceptive is inserted into the vaginal canal within 72 hours before or after intercourse, within 48 hours before or after intercourse, within 36 hours before or after intercourse, within 24 hours before or after intercourse, within 12 hours before or after intercourse, or during intercourse.
